(19) United States
(12) Patent Application Publication
(10) Pub. No.: US 2017/0133603 A1
(43) Pub. Date: May 11, 2017

(54) COMPOUND FOR PHOSPHORESCENT HOST AND ORGANIC LIGHT-EMITTING ELEMENT COMPRISNG SAME

(71) Applicant: BYUCKSAN PAINT & COATINGS CO. LTD, Seoul (KR)

(72) Inventors: KukSoung JOUNG, Seoul (KR); KyuSoung KIM, Gyeonggi-do (KR)

(73) Assignee: BYUCKSAN PAINT & COATINGS CO. LTD, Seoul (KR)

(21) Appl. No.: 15/319,009

(22) PCT Filed: Mar. 24, 2015
(86) PCT No.: PCT/KR2015/002869

§ 371 Date: Dec. 15, 2016

(30) Foreign Application Priority Data
Jul. 1, 2014 (KR) 10-2014-0082046

(51) Int. Cl. H01L 51/00; C07D 519/00; C07D 409/14; C09K 11/02; C09K 11/06

(52) U.S. Cl. CPC H01L 51/0072; C09K 11/02; C09K 11/06; H01L 51/0074; C07D 409/14; C07D 519/00; H01L 51/5016

(57) ABSTRACT

Disclosed is a phosphor host compound having a donor-acceptor-donor (D-A-D) structure wherein a thioxanthene compound occupies the acceptor core responsible for the mother skeleton of the structure, and carbazole or carboline compounds serve as the peripheral donors connected to the central atom opposite to the sulfone radical in the mother skeleton.

### BACKGROUND OF THE INVENTION

1. Field of the Invention

The present invention relates to a compound for phosphorescent host and organic light-emitting element comprising same. More particularly, the present invention relates to a phosphor host compound that has a donor-acceptor-donor (D-A-D) structure with a wide band gap, and an organic light emitting device comprising the same, which can emit blue light with high luminous efficiency.

2. Description of the Related Art

An electroluminescent device (EL device) is a self-luminous display device characterized by a wide viewing angle, a high contrast, and a fast response speed.

EL devices are classified into inorganic and organic EL devices according to the materials used for an emitting layer. Organic EL devices have great advantages over inorganic EL devices in terms of brightness, driving voltage, and response speed, and in that they can realize polychromaticity.

On the whole, the structure of an organic EL device is such that an anode, a hole transport layer, a luminescent layer, an electron transport layer, and a cathode are sequentially formed on a substrate. In this regard, the hole transport layer, the luminescent layer, and the electron transport layer exist as respective thin films made of organic compounds.

With such a structure, an organic EL device is principally operated as follows. When a voltage is applied between the anode and the cathode, a hole injected from the anode migrates to the emitting layer through the hole transport layer. Meanwhile, an electron is released from the cathode and moves through the electron transport layer toward the luminescent layer. In the luminescent zone, the carriers recombine to produce an exciton. When the exciton returns to the ground state from the excited state, the molecule of the luminescent layer emits light, forming an image. According to luminescence mechanism, luminescent materials used in the luminescent layer can be divided into fluorescents and phosphorescents, which utilize excitons in a singlet state and a triplet state, respectively. Generally, a phosphorescent material has an organic-inorganic compound structure containing a heavy atom that functions to enhance the transition of excitons in a triplet state, which is a forbidden state, but is allowed to undergo the transition, thus emitting phosphorescent light. Capable of producing triplet excitons at 75% probability, a phosphorescent material has much higher luminous efficiency than a fluorescent material, which utilizes singlet excitons at 25% probability.

A luminescent layer that uses a phosphorescent material is composed of a host material, and a dopant that emits light with the transition of energy from the host material. There are various materials, including iridium compounds, reported as dopant materials. Research at Princeton University, Southern California University, etc. has been conducted on organic luminescent materials using iridium compounds, and suggested various phosphorescent materials of iridium, platinum metal compounds, but there is still a demand for a material that exhibits better luminescent properties and is of higher stability.

Leading to the present invention, intensive and thorough research into host materials resulted in the finding that when a host compound is designed to have a donor-acceptor-donor (D-A-D) structure wherein a thioxanthene compound occupies the acceptor core responsible for the mother skeleton of the structure, and carbazole or carboline compounds serve as the peripheral donors connected to the central atom opposite to the sulfone radical in the mother skeleton and wherein either or both of the donors are symmetrically or asymmetrically substituted with an alkyl radical or penta- or hexagonal ring derivative, the host compound has an amorphous property with a wide band gap and high triplet energy (ET), and exhibits a high glass transition temperature (Tg), long life span, and high thermal stability.

### RELATED ART DOCUMENT

(Patent Document 1) Korean Patent Unexamined Application Publication No. 10-2006-0113935 (titled “Organic Element for Electroluminescent Devices”, issued on Nov. 3, 2006)

### SUMMARY OF THE INVENTION

It is therefore a first object of the present invention to provide a phosphor host compound that has a donor-acceptor-donor (D-A-D) structure guaranteeing a wide band gap.

It is another object of the present invention to provide an organic light emitting device using a phosphor host compound that has a donor-acceptor-donor (D-A-D) structure guaranteeing a wide band gap.

In order to accomplish the first object, an aspect of the present invention addresses a phosphor host compound, represented by the following Chemical Formula 1. The phosphor host compound may be a blue phosphor host compound.

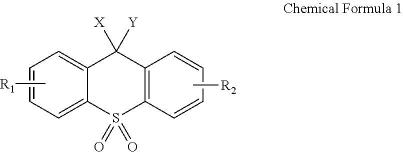

wherein,

X and Y are independently selected from among a carbazole group and a carboline group, and

R1 and R2, which may be the same or different substituted or unsubstituted functional radicals, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C1-C14 alkylcarbonyl or substituted or unsubstituted C7-C30 arylcarbonyl, or C1-C14 alkylthio.

In accordance with another aspect of the present invention, the second object may be accomplished by providing an organic light emitting device, comprising an organic film between a pair of electrodes, wherein the organic film comprises a phosphor host compound represented by the following Chemical Formula 1:

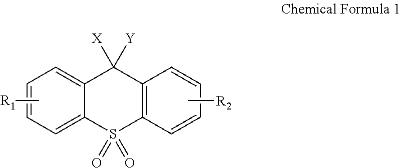

wherein,

X and Y are independently selected from among a carbazole group and a carboline group, and

R1 and R2, which may be the same or different substituted or unsubstituted functional radicals, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C1-C14 alkylcarbonyl or substituted or unsubstituted C7-C30 arylcarbonyl, or C1-C14 alkylthio.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic cross sectional view illustrating a structure of an organic light emitting device according to one exemplary embodiment of the present invention; and

FIG. 2 is NMR spectrum of 9,9-bis(9-ethyl-9H-carbazol-3-yl)-9H-thioxanthene 10,10-dioxide synthesized according to one exemplary embodiment of the present invention.

### DESCRIPTION OF THE PREFERRED EMBODIMENTS

Below, a detailed description will be given of the present invention.

The present invention addresses a phosphor host compound designed to have a donor-acceptor-donor (D-A-D) structure with a great HOMO-LUMO gap, wherein a thioxanthene compound covers a mother skeleton functioning as the acceptor core, while carbazole and/or carboline compounds serve as the two donor moieties that are both positioned at the central atom opposite to the sulfone radical in the mother skeleton. The D-A-D structure may be symmetrical when the same donor moieties are positioned at the central atom opposite to the sulfone radical in the mother skeleton. The D-A-D structure may be symmetrical or asymmetrical. When the two donor moieties are respectively occupied by a carbazole compound and a carboline compound, the D-A-D structure is asymmetric. Although either carbazole compounds or carboline compounds cover both the donor moieties, the D-A-D structure may be asymmetric as the carbazoline or caboline compounds are differently substituted at the intramolecular amine atom or at the cyclic ring with an alkyl radical or a penta- or hexagonal ring derivative.

In detail, the phosphor host compound of the present invention has a structure represented by the following Chemical Formula 1:

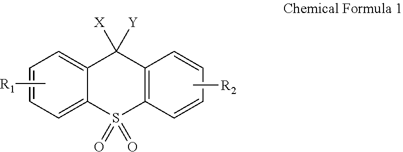

wherein, X and Y are independently selected from among a carbazole group and a carboline group. The carboline group may be preferably selected from the group consisting of α-carboline, β-carboline, γ-carboline, and δ-carboline.

In Chemical Formula 1, R1 and R2, which may be the same or different substituted or unsubstituted functional radicals, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C1-C14 alkylcarbonyl or substituted or unsubstituted C7-C30 arylcarbonyl, or C1-C14 alkylthio.

Representative examples of the compounds of Chemical Formula 1 according to the present invention include those where X and Y both are carboazole, α-carboline, β-carboline, γ-carboline, or δ-carboline, as represented by the following Chemical Formulas 2 to 6:

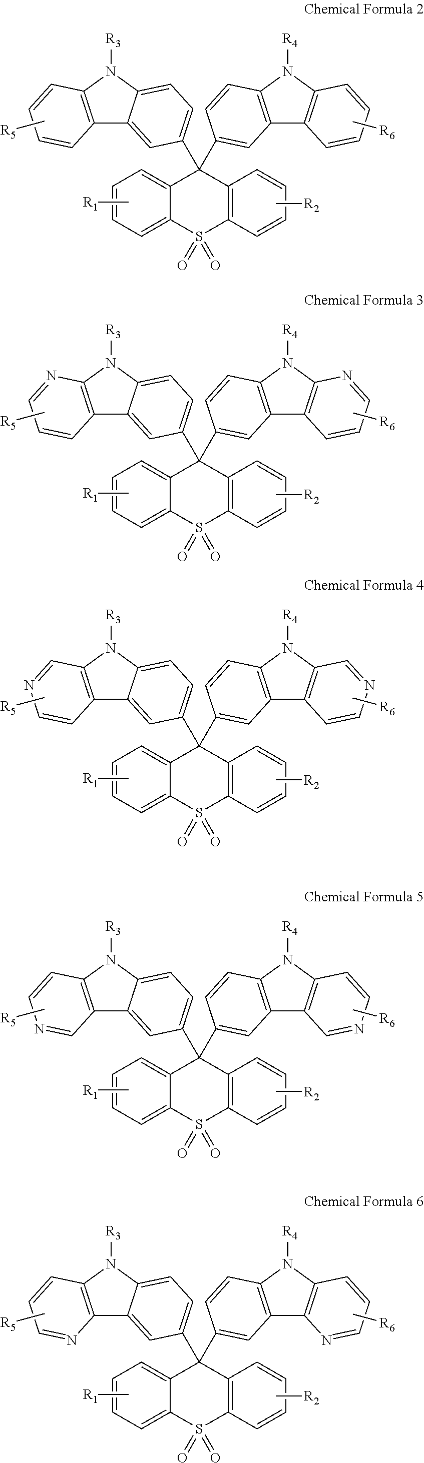

In Chemical Formula 2 to 6, R1 to R6, which may be the same or different substituted or unsubstituted functional groups, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C1-C14 alkylcarbonyl, substituted or unsubstituted C7-C14 arylcarbonyl or C1-C14 alkylthio, and adjacent ones of them may be combined to form a ring.

Preferred examples of compounds of Chemical Formula 1 or 6 may be represented by the following Chemical Formula 7 to 11:

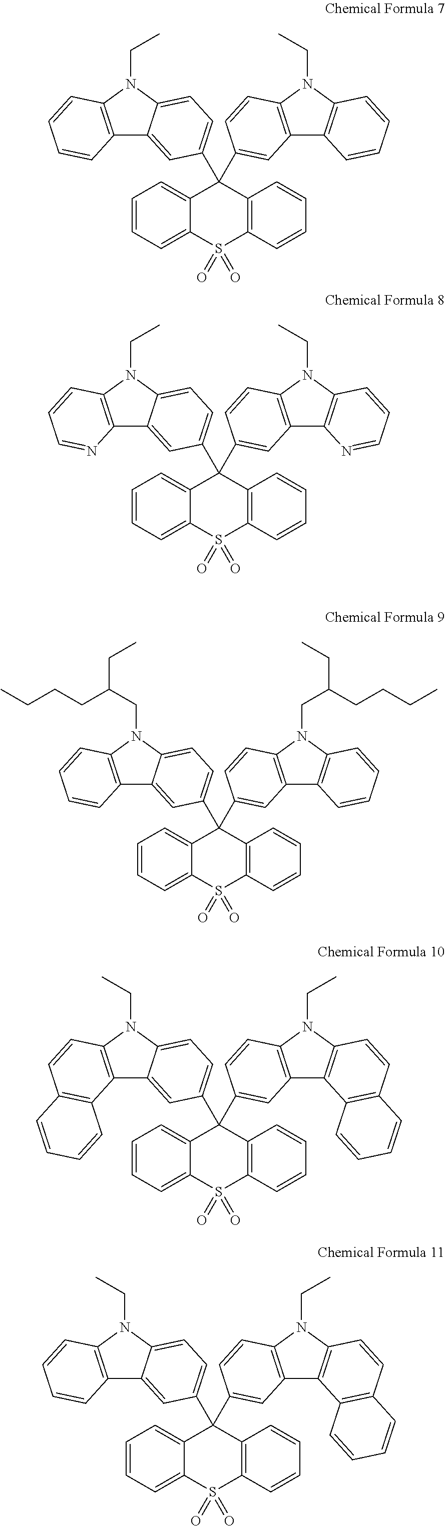 [SMILES: CCn1c2ccccc2c2cc(C3(c4ccc5c(c4)c4ccccc4n5CC)c4ccccc4S(=O)(=O)c4ccccc43)ccc21 ; CCn1c2ccc(C3(c4ccc5c(c4)c4ncccc4n5CC)c4ccccc4S(=O)(=O)c4ccccc43)cc2c2ncccc21 ; CCCCC(CC)Cn1c2ccccc2c2cc(C3(c4ccc5c(c4)c4ccccc4n5CC(CC)CCCC)c4ccccc4S(=O)(=O)c4ccccc43)ccc21 ; CCn1c2ccc(C3(c4ccc5c(c4)c4c6ccccc6ccc4n5CC)c4ccccc4S(=O)(=O)c4ccccc43)cc2c2c3ccccc3ccc21 ; CCn1c2ccccc2c2cc(C3(c4ccc5c(c4)c4c6ccccc6ccc4n5CC)c4ccccc4S(=O)(=O)c4ccccc43)ccc21]

As used herein, the term “unsubstituted alkyl” or “unsubstituted alkoxy” refers to a corresponding radical with an alkyl moiety containing 1 to 14 carbon atoms (identified as, for example, methyl, ethyl, propyl, butyl, pentyl, hexyl, heptyl, octyl, nonyl, decyl, undecyl, dodecyl, tridecyl, tetradecyl, pentadecyl, etc. or isomers thereof), and the term “substituted alkyl” or “substituted alkoxy” has the same meaning as in the “unsubstituted” counterpart with the exception that at least one hydrogen atom of the alkyl moiety is substituted by a halogen atom, hydroxy, nitro, cyano, amino, amidino, hydrazine, hydrazone, carboxyl or a salt thereof, sulfonic acid or a salt thereof, phosphoric acid or a salt thereof, C1-C14 alkyl, C2-C14 alkenyl, C2-C14 alkynyl, C6-C14 aryl, C7-C14 arylalkyl, C2-C14 heteroaryl, or C3-C14 heteroalkyl.

As used herein, the term “unsubstituted alkenyl” means a C1 to C14 aliphatic hydrocarbon with at least one double bond (for example, ethene, protene, butene, pentene, hexane, etc., and isomers thereof), and the term “substituted alkenyl” has the same meaning as the unsubstituted alkenyl, with the exception that at least one hydrogen atom on the alkenyl framework is substituted as in the substituted alkyl.

The “unsubstituted aryl” means a functional group or substituent derived from one or more aromatic rings of 6 to 14 carbon atoms, where two or more rings, if any, may be connected to each other in a pendent manner or may fused to each other while “substituted aryl” has the same meaning as the “unsubstituted aryl” with the exception that at least one hydrogen atom on the aryl framework is substituted as in the substituted alkyl.

The term “unsubstituted arylalkyl” has the same meaning as the “unsubstituted aryl,” with the exception that a part of the aryl framework is substituted by lower alkyl, for example, methyl, ethyl, propyl, etc. The term “substituted arylalkyl” has the same meaning as the unsubstituted arylalkyl, with the exception that at least one hydrogen atom on the arylalkyl framework is substituted as in the substituted alkyl.

The term “unsubstituted aryloxy” means aryl substituted with an oxygen atom as in, for example, phenyloxy, naphthalenoxy, diphenyloxy, etc. The term “substituted aryloxy” has the same meaning as the unsubstituted aryloxy, with the exception that at least one hydrogen atom on the aryloxy framework is substituted as in the substituted alkyl.

As used herein, “unsubstituted heteroaryl” means a 2- or 14-membered, monovalent monocyclic or divalent bicyclic aromatic compound containing 1, 2, or 3 heteroatoms selected from among N, O, P, and S, the remaining membered atoms being carbon, examples being thienyl, pyridyl, furyl, etc. The “substituted heteroaryl” is the same as the unsubstituted heteroaryl, with the exception that at least one hydrogen atom on the heteroaryl framework is substituted as in the substituted alkyl.

The term “unsubstituted heteroarylalkyl” has the same meaning as the “unsubstituted heteroaryl,” with the exception that a part of the heteroaryl framework is substituted by lower alkyl while “substituted arylalkyl” has the same meaning as the unsubstituted heteroarylalkyl, with the exception that at least one hydrogen atom on the heteroarylalkyl framework is substituted as in the substituted alkyl.

The term “unsubstituted heteroaryloxy” means heteroaryl substituted with an oxygen atom. The term “substituted heteroaryloxy” has the same meaning as the unsubstituted aryloxy, with the exception that at least one hydrogen atom on the heteroaryloxy framework is substituted as in the substituted alkyl.

In addition, “unsubstituted cycloalkyl” means monovalent monocyclic containing 4 to 14 carbon atoms, as exemplified by cyclohexyl, cyclopentyl, etc. The term “substituted cycloalkyl” has the same meaning as the unsubstituted cycloalkyl, with the exception that at least one hydrogen atom on the cycloalkyl framework is substituted as in the substituted alkyl.

As used herein, the term “unsubstituted heterocycloalkyl” means a 1- or 30-membered, monovalent monocyclic compound containing 1, 2, or 3 heteroatoms selected from among N, O, P, and S, the remaining atoms being carbon, with the exception that a part of the heterocyclic framework is substituted by lower alkyl while “substituted heterocycloalkyl” has the same meaning as the unsubstituted heterocycloalkyl, with the exception that at least one hydrogen atom on the heterocycloalkyl framework is substituted as in the substituted alkyl.

Concrete examples of the “unsubstituted alkylcarbonyl” include acetyl, ethylcarbonyl, isopropylcarbonyl, phenylcarbonyl, naphthalenecarbonyl, diphenylcarbonyl, and cyclohexylcarbonyl while “substituted alkylcarbonyl” is the same as the unsubstituted alkylcarbonyl, with the exception that at least one hydrogen atom on the alkylcarbonyl framework is substituted as in the substituted alkyl.

Compounds falling within the scope of “unsubstituted arylcarbonyl” may be exemplified by phenylcarbonyl, naphthalenecarbonyl, diphenylcarbonyl, etc., and the term “substituted arylcarbonyl” has the same meaning as the unsubstituted arylcarbonyl, with the exception that at least one hydrogen atom on the arylcarbonyl framework is substituted as in the substituted alkyl.

Taking a donor-acceptor-donor structure, the phosphor host compound, represented by Chemical Formula 1, according to the present invention has a band gap of 3.0 eV or higher and thus can release high energy, emitting phosphorescent light with high luminous efficiency.

Below, an explanation will be given of the structure and fabrication of an organic light emitting device utilizing the phosphor host compound of the present invention.

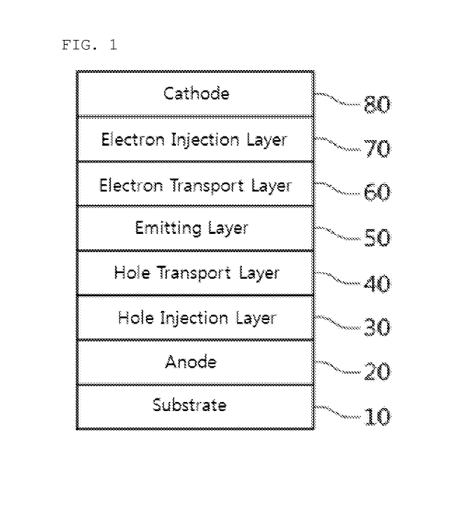

The organic light emitting device according to the present invention may adopt a typical structure of electroluminescent devices, and its architecture may be modified if necessary. In principle, the organic light emitting device comprises a first electrode (anode) and a second electrode (cathode) with an organic membrane (luminescent layer) sandwiched therebetween. As needed, it may further comprise a hole injection layer, a hole transport layer, a hole blocking layer, an electron injection layer, and/or an electron transport layer. With regard to the structure of a luminescent device according to an exemplary embodiment of the present invention, reference is made to FIG. 1.

As shown in FIG. 1, the organic light emitting device in accordance with an exemplary embodiment of the present invention is structured to include an anode 20 and a cathode 80, with an emitting layer 50 interposed therebetween. In the device, a hole injection layer 30 and a hole transport layer 40 are sandwiched between the anode 20 and the emitting layer while an electron transport layer 60 and an electron injection layer 70 lie between the emitting layer 50 and the cathode 80.

The organic light emitting device, shown in FIG. 1, according to an exemplary embodiment of the present invention can be fabricated in the following processes, which are illustrative, but not limitative.

First, an anodic material is applied to a top of a substrate 10 to form an anode 20. So long as it is typically used in the art, any substrate 10 may be employed. Particularly preferred is a glass or transparent plastic substrate that is superior in terms of transparency, surface smoothness, ease of handling, and water resistance. Examples of the anode material include, but are not limited, to indium tin oxide (ITO), tin oxide (SnO₂), and zinc oxide (ZnO), which are transparent and exhibit excellent conductivity.

Optionally, a hole injection layer (HIL) 30 is formed on the anode 20. Formation of the hole injection layer can be achieved by a typical method such as vacuum deposition or spin coating. No particular limitations are imposed on materials for the hole injection layer, and CuPc (copper phthalocyanine) or IDE 406 (Idemitsu Kosan) may be used.

Next, a hole transport layer (HTL) 40 is formed on the hole injection layer 30, using a typical method such as vacuum deposition or spin coating. Examples of the material for the hole transport layer include N,N′-diphenyl-N,N′-bis(1-naphthyl)-1,1′-biphenyl-4,4′-diamine(NPB) N,N′-bis(3-methylphenyl)-N,N′-diphenyl-[1,1-biphenyl]-4,4′-diamine (TPD), N,N′-di(naphthalen-1-yl)-N,N′-diphenylbenzidine, and N,N′-di(naphthalen-1-yl)-N,N′-diphenyl-benzidine (α-NPD), but are not limited thereto.

Subsequently, an emitting layer (EML) 50 is formed on the hole transport layer 40. When the emitting layer is formed, at least one of the phosphor host compounds of the present invention is used as a luminescent host in the layer. In addition, the emitting layer may be a monolayer structure or a multilayer structure, such as bi- or higher layer structure. The compound of Chemical Formula 1 may be used alone or in combination with other compounds known in the art, for example, blue phosphorescent dopants (iridium compounds such as FIrppy or FIrpic). The phosphor host compound of the present invention may be used in an amount of 1 to 95 weight %, based on the total weight of the emitting layer.

For the formation of the emitting layer, the phosphor host compound may be applied by vacuum deposition or may be deposited in a wet process such as spin coating, or by laser induced thermal imaging (LITI).

On the emitting layer 50, optionally, a hole blocking layer may be formed for blocking the migration of the excitons formed in the luminescent material to the electron transport layer 60 or for blocking the migration of holes to the electron transport 60. No particular limitations are imposed on materials for the hole blocking layer, and phenanthroline compounds (for example, BCP) may be used. This material may be layered on the emitting layer by vacuum deposition or spin coating.

Alternatively, an electron transport layer (ETL) 60 may be formed on the emitting layer 50, using a vacuum deposition process or a spin coating process. Materials for the electron transport layer include, but are not limited to, TBPI, and aluminum complexes (e.g., Alq3(tris(8-quinolinolato)-aluminum)).

On the electron transport layer 60, an electron injection layer (EIL) 70 may be formed by vacuum deposition or spin coating. No particular limitations are imposed on a material for the electron injection layer 70, and it may be formed of LiF, NaCl, or CsF.

Afterwards, a cathode 80 is formed on the electron injection layer 70 by vacuum deposition to complete a luminescent device. In this context, a metal such as lithium (Li), magnesium (Mg), aluminum (Al), aluminum-lithium (Al—Li), calcium (Ca), magnesium-indium (Mg—In), or magnesium-silver (Mg—Ag) may be deposited to form the cathode.

In an exemplary embodiment of the present invention, the organic light emitting device has the stack structure shown in FIG. 1. As needed, one or two intermediate layers, for example, a hole blocking layer, may be further formed. The thickness of each of the layers in the luminescent device may be determined within the ranges generally accepted in the art.

A better understanding of the present invention may be obtained through the following examples which are set forth to illustrate, but are not to be construed as limiting the present invention.

### Synthesis Example 1

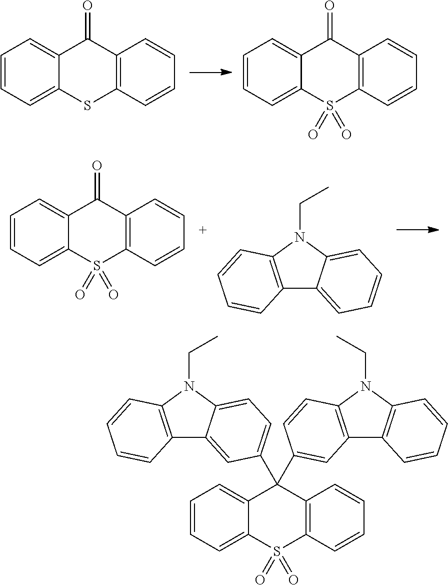

### Synthesis of 9H-Thioxanthen-9-one 10,10-dioxide

After being stirred for 30 min, of 9-H-thioxanthen-9-one (10 g, 47 mmol) in 200 ml of glacial acetic acid was mixed with hydrogen peroxide (1.8 g, 53 mmol), and refluxed for 2 hrs. After completion of the reaction, the resulting reaction mixture was stirred at room temperature for 2 hrs, and the precipitate thus formed was washed with n-hexane, and dried to afford the title compound. 8.6 g (yield: 75%).

### Synthesis of 9,9-Bis(9-ethyl-9H-carbazol-3-yl)-9H-thioxanthen 10,10-dioxide

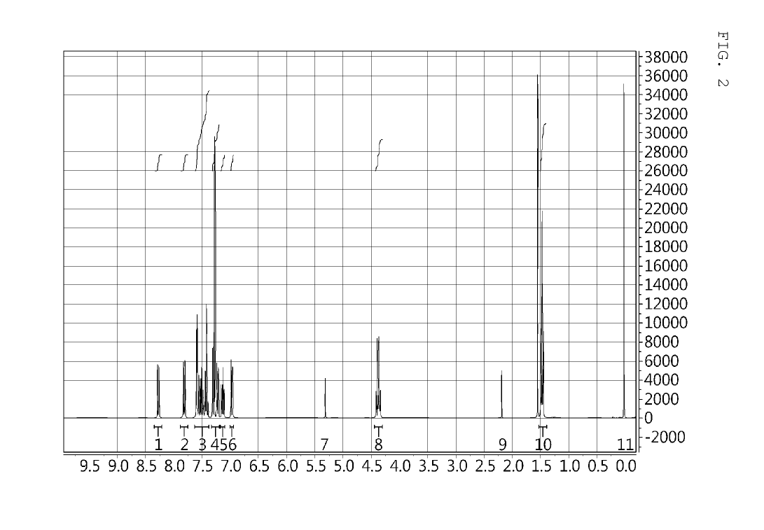

In a reactor, 9H-thioxanthen-9-one 10,10-dioxide (8 g, 32.75 mmol), obtained above, and 9-ethyl-9H-carbazole (19.18 g, 98.25 mmol) were placed and heated to 80° C. under a nitrogen atmosphere. To this reaction mixture, a solution of phosphorus pentoxide (23.24 g, 163.75 mmol) in methanesulfonic acid (277 g) was slowly added over 1 hr, and reacted for 24 hrs. When the reaction was completed, the reaction mixture was cooled to room temperature, and mixed with MeOH (600 ml) for 1 hr while stirring. The precipitate thus formed was filtered and washed with MeOH. The precipitate was heated in acetone (300 ml) for 1 hr under reflux, and then was further stirred for 1 hr at room temperature and filtered. The filtrate was purified by column chromatography using a mixture of 1:1 dichloromethane:petroleum ether as an eluting solution to afford the title compound 9,9-bis(9-ethyl-9H-carbazol-3-yl)-9H-thioxanthene 10,10-dioxide. 10.9 g (yield: 54%). An NMR spectrum of the compound is given in FIG. 2.

On the NMR spectrum, peak 1 was read at 8.29, 8.27, and 8.26, peak 2 at 7.81, and 7.83, peaks 3 to 6 at 6.95, 6.96, 6.98, 6.99, 7.13, 7.21, 7.21, 7.24, 7.24, 7.28, 7.29, 7.32, 7.42, 7.42, 7.44, 7.45, 7.51, 7.51, 7.56, 7.56, 7.58, 7.59, and 7.60, peak 7 at 5.32, peak 8 at 4.34, 4.37, 4.39, and 4.42, peak 9 at 2.19, peak 10 at 1.45, 1.48, 1.50, and 1.56, and peak 11 at 0.03.

### Synthesis Example 2

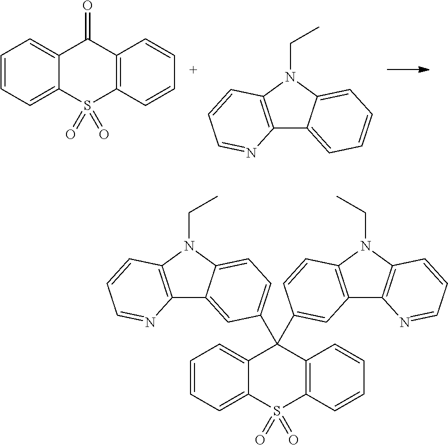

### Synthesis of 9,9-Bis(5-ethyl-5H-pyrido[3,2-b]indol-8-yl)-9H-thioxanthene 10,10-dioxide

In a reactor, 9H-thioxanthen-9-one 10,10-dioxide (10 g, 40.94 mmol) and 5-ethyl-5H-pyrido[3,2-b]indole (24.1 g, 122.8 mmol) were placed and heated to 90° C. under a nitrogen atmosphere. To this reaction mixture, a solution of phosphorus pentoxide (29 g, 0.205 mmol) in methanesulfonic acid (345 g) was slowly added over 1 hr, and reacted for 36 hrs. When the reaction was completed, the reaction mixture was cooled to room temperature, and mixed with MeOH (700 ml) for 1 hr while stirring. The precipitate thus formed was filtered and washed with MeOH. The precipitate was heated in acetone (400 ml) for 1 hr under reflux, and then was further stirred for 1 hr at room temperature, washed with acetone, and filtered. The filtrate was purified by column chromatography using a mixture of 1:2 dichloromethane:ethylacetate as an eluting solution to afford the title compound 9,9-bis(5-ethyl-5H-pyrido[3,2-b] indol-8-yl)-9H-thioxanthene 10,10-dioxide. 12.73 g (yield: 50.3%).

### Synthesis Example 3

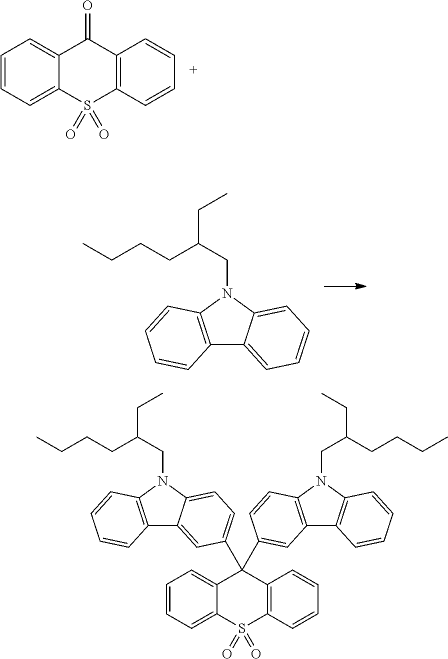

### Synthesis of 9,9-Bis(9-(2-ethylhexyl)-9H-carbazol-3-yl)-9H-thioxanthene 10,10-dioxide

In a reactor, 9H-thioxanthen-9-one 10,10-dioxide (5 g, 20.47 mmol) and 9-(2-ethylhexyl)-9H-carbazole (17.16 g, 61.41 mmol) were placed and heated to 80° C. under a nitrogen atmosphere. To this reaction mixture, a solution of phosphorus pentoxide (14.5 g, 0.102 mmol) in methanesulfonic acid (173 g) was slowly added over 1 hr, and reacted for 18 hrs. When the reaction was completed, the reaction mixture was cooled to room temperature, and mixed with MeOH (500 ml) for 1 hr while stirring. The precipitate thus formed was filtered and washed with MeOH. The precipitate was heated in acetone (300 ml) for 1 hr under reflux, and then was further stirred for 1 hr at room temperature, washed with acetone, and filtered. The filtrate was purified by column chromatography using a mixture of 1:1 dichloromethane:petroleum ether as an eluting solution to afford the title compound 9,9-bis(9-(2-ethylhexyl)-9H-carbazol-3-yl)-9H-thioxanthene 10,10-dioxide. 9 g (yield: 56%).

### Synthesis Example 4

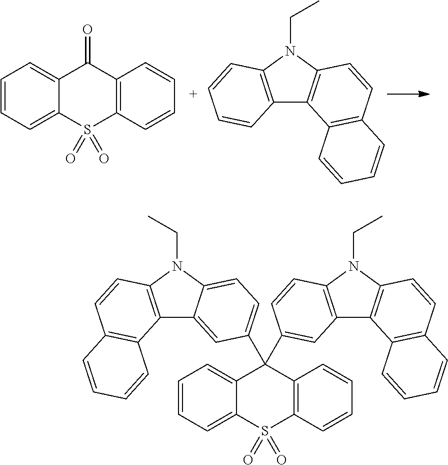 [SMILES: O=C1c2ccccc2S(=O)(=O)c2ccccc21 ; CCn1c2c(c3c4ccccc4ccc31)C=C[C+]=C2 ; CCn1c2ccc(C3(c4ccc5c(c4)c4c6ccccc6ccc4n5CC)c4ccccc4S(=O)(=O)c4ccccc43)cc2c2c3ccccc3ccc21]

### Synthesis of 9,9-Bis(7-ethyl-7H-benzo[c]carbazol-10-yl)-9H-thioxanthene 10,10-dioxide

In a reactor, 9H-thioxanthen-9-one 10,10-dioxide (10 g, 40.94 mmol) and 7-ethyl-7H-benzo[c]carbazole (30.13 g, 122.8 mmol) were placed and heated to 100° C. under a nitrogen atmosphere. To this reaction mixture, a solution of phosphorus pentoxide (29 g, 0.205 mmol) in methanesulfonic acid (345 g) was slowly added over 1 hr, and reacted for 20 hrs. When the reaction was completed, the reaction mixture was cooled to room temperature, and mixed with MeOH (900 ml) for 1 hr while stirring. The precipitate thus formed was filtered and washed with MeOH. The precipitate was heated in acetone (700 ml) for 1 hr under reflux, and then was further stirred for 1 hr at room temperature, washed with acetone, and filtered. The filtrate was purified by column chromatography using a mixture of 1:3 dichloromethane:petroleum ether as an eluting solution to afford the title compound 9,9-bis(7-ethyl-7H-benzo[c]carbazol-10-yl)-9H-thioxanthen 10,10-dioxide. 13.5 g (yield. 46.1%).

### Synthesis Example 5

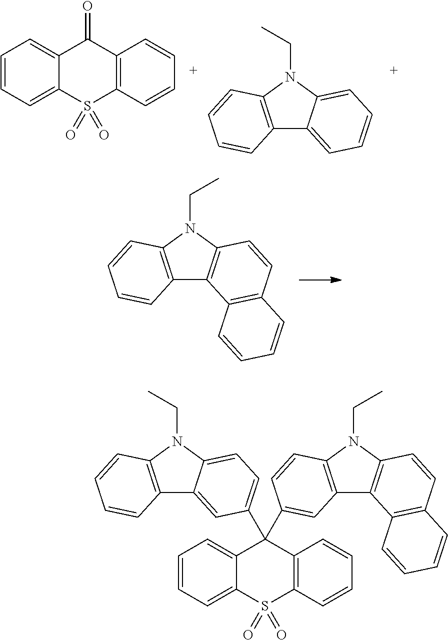

### Synthesis of 9-(7-Ethyl-7H-benzo[c]carbazol-10-yl)-9-(9-ethyl-9H-carbazol-3-yl)-9H-thioxanthene 10,10-dioxide

In a reactor, 9H-thioxanthen-9-one 10,10-dioxide (8 g, 32.75 mmol), 9-ethyl-9H-carbazole (9.6 g 49.13 mmol), and 7-ethyl-7H-benzo[c]carbazole (12.05 g, 49.13 mmol) were placed, and heated to 90° C. under a nitrogen atmosphere. To this reaction mixture, a solution of phosphorus pentoxide (23.24 g, 163.75 mmol) in methanesulfonic acid (277 g) was slowly added over 1 hr, and reacted for 24 hrs. When the reaction was completed, the reaction mixture was cooled to room temperature, and mixed with MeOH (800 ml) for 1 hr while stirring. The precipitate thus formed was filtered and washed with MeOH. The precipitate was heated in acetone (700 ml) for 1 hr under reflux, and then was further stirred for 1 hr at room temperature, washed with acetone, and filtered. The filtrate was purified by column chromatography using a mixture of 1:2 dichloromethane:petroleum ether as an eluting solution to afford the title compound 9-(7-ethyl-7H-benzo[c]carbazol-10-yl)-9-(9-ethyl-9H-carbazol-3-yl)-9H-thioxanthene 10,10-dioxide. 8.3 g (yield: 38%).

### Test Example 1

9,9-Bis(9-ethyl-9H-carbazol-3-yl)-9H-thioxanthene 10,10-dioxide, obtained in Synthesis Example 1, was evaluated for physical properties, and the results are summarized in Table 1, below.

TABLE 1

| | UV | PL | | | Band | | | | |
|---|---|---|---|---|---|---|---|---|---|
| | max | max | HOMO | LUMO | gap | Ti | T_{ID} | Tm | Tg |
| Property | (nm) | (nm) | (eV) | (eV) | (eV) | (eV) | (° C.) | (° C.) | (° C.) |
| Synthesis | 303, | 377 | 5.64 | 2.22 | 3.42 | 2.93 | 373 | 336 | 217 |
| Example 1 | 339, | | | | | | | | |
| | 353 | | | | | | | | |

UVₘₐₓ: absorption wavelength of material, measured by spectrometry and cyclic voltammetry
PLₘₐₓ: photoluminescence wavelength of material, measured by spectrometry and cyclic voltammetry
HOMO, LUMO, Band gap: electrical properties of material, measured by spectrometry and cyclic voltammetry (for blue light, a wide band gap of >3.0 eV is required).
T₁: triplet energy of material, measured by spectrometry and cyclic voltammetry (phosphorescence measured at 77K)
TID: degradation temperature (measured by TGA)
Tₘ: melting point
Tg: glass transition temperature

The triplet energy (T₁) of the host material synthesized in Synthesis Example 1 in accordance with the present invention is higher than that (2.7 eV) of a typical dopant (e.g., Firpic), thus allowing more effective energy release. In addition, the synthesized compound is thermally stable with a Tg of 170 Celsius degrees.

### Example 1

An ITO substrate was patterned to make an emitting area of 3 mm×3 mm, and then rinsed. The substrate was placed in a vacuum chamber which was then vacuumed to a base pressure of 1×10⁻⁶ torr. On the anode ITO, a hole transport layer was formed by depositing an NPB film to a thickness of 40 nm, followed by forming a 20-nm-thick emitting layer with 9,9-bis(9-ethyl-9H-carbazol-3-yl)-9H-thioxanthene 10,10-dioxide, synthesized in Synthesis Example 1, and the dopant [FCNIr] at a doping concentration of 11% on the hole transport layer. Thereafter, TPBI was vacuum deposited on the emitting layer to form an electron transport layer 50 nm thick. LiF was deposited on the emitting layer to form an electron injection layer 1.0 nm thick, and Al was deposited to a thickness of 500 nm to form a cathode on the electron injection layer. The organic light emitting device thus obtained was evaluated for electrical and optical properties, and the results are summarized in Table 2, below.

### Example 2

An organic light emitting device was fabricated in the same manner as in Example 1, with the exception that 9,9-bis(5-ethyl-5H-pyrido[3,2-b]indol-8-yl)-9H-thioxanthene 10,10-dioxide, synthesized in Synthesis Example 2, was used, instead of the compound synthesized in Synthesis Example 1, as a host of the emitting layer. Electrical and optical properties of the device were measured, and the results are summarized in Table 2, below.

### Comparative Example 1

An organic light emitting device was fabricated in the same manner as in Example 1, with the exception that mCP (1,3-bis (N-carbazolyl)benzene was used, instead of the compound synthesized in Synthesis Example 1, as a host of the emitting layer. Electrical and optical properties of the device were measured, and the results are summarized in Table 2, below.

TABLE 2

| | | Current | Current | Power | Quantum | |
|---|---|---|---|---|---|---|
| Ex. | Volt. | Density | Effici. | Effici. | Effici. | Brightness |
| No. | [V] | [mA/cm²] | Cd/A | [Lm/W] | [%] | [Cd/m²] |
| Ex. 1 | 6.29 | 6.9 | 14.22 | 7.09 | 8.21 | 1000 |
| Ex. 2 | 6.67 | 14.26 | 6.9 | 3.23 | 3.99 | 1000 |
| C. Ex. 1 | 6.65 | 14.5 | 6.77 | 3.19 | 3.863 | 1000 |

### DESCRIPTION OF REFERENCE NUMERALS IN THE DRAWINGS

10: Substrate
20: Anode
30: Hole Injection Layer
40: Hole Transport Layer
50: Emitting Layer
60: Electron Transport Layer
70: Electron Injection Layer
80: Cathode

Designed to have a donor-acceptor-donor (D-A-D) wherein a thioxanthene compound occupies the acceptor core responsible for the mother skeleton of the structure, and carbazole or carboline compounds serve as the peripheral donors connected to the central atom opposite to the sulfone radical in the mother skeleton, as described hitherto, the phosphor host compound according to the present invention has an amorphous property with a wide band gap and high triplet energy (ET), and exhibits a high glass transition temperature (Tg), long life span, and high thermal stability. Also, a luminescent device utilizing the phosphor host compound can show excellent luminescent properties and stability.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A phosphor host compound, represented by the following Chemical Formula 2: 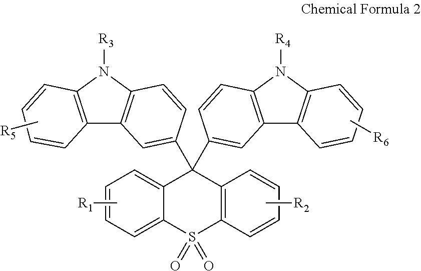
wherein,
R1 to R6, which may be the same or different substituted or unsubstituted functional groups, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C2-C14 alkylcarbonyl, substituted or unsubstituted C7-C14 arylcarbonyl or C1-C14 alkylthio, or
adjacent ones of R1 to R6 may be combined to form a ring.

2. An organic light emitting device, comprising an organic film between a pair of electrodes, wherein the organic film comprises the phosphor host compound of claim 1.

3. The organic light emitting device of claim 2, wherein the organic film is an emitting layer.

4. A phosphor host compound, represented by the following Chemical Formula 6: 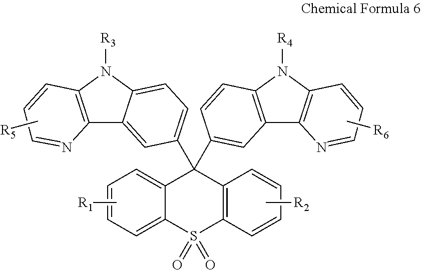
wherein,
R1 to R6, which may be the same or different substituted or unsubstituted functional groups, are independently a hydrogen atom, cyano, hydroxy, thiol, a halogen atom, substituted or unsubstituted C1-C14 alkyl, substituted or unsubstituted C1-C14 alkoxy, substituted or unsubstituted C2-C14 alkenyl, substituted or unsubstituted C6-C14 aryl, substituted or unsubstituted C6-C14 arylalkyl, substituted or unsubstituted C6-C14 aryloxy, substituted or unsubstituted C2-C14 heteroaryl, substituted or unsubstituted C2-C14 heteroarylalkyl, substituted or unsubstituted C2-C14 heteroaryloxy, substituted or unsubstituted C5-C14 cycloalkyl, substituted or unsubstituted C2-C14 heterocycloalkyl, substituted or unsubstituted C1-C14 alkylcarbonyl, substituted or unsubstituted C7-C14 arylcarbonyl or C1-C14 alkylthio, or
adjacent ones of R1 to R6 may be combined to form a ring.

5. An organic light emitting device, comprising an organic film between a pair of electrodes, wherein the organic film comprises the phosphor host compound of claim 4.

6. The organic light emitting device of claim 5, wherein the organic film is an emitting layer.

7. A phosphor host compound, represented by the following Chemical Formula 7:

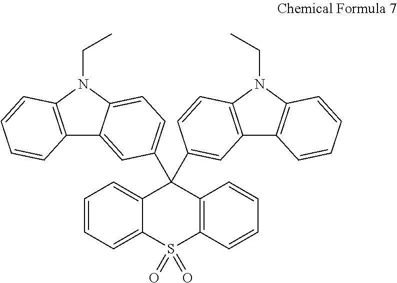 [SMILES: CCn1c2ccccc2c2cc(C3(c4ccc5c(c4)c4ccccc4n5CC)c4ccccc4S(=O)(=O)c4ccccc43)ccc21]

8. An organic light emitting device, comprising an organic film between a pair of electrodes, wherein the organic film comprises the phosphor host compound of claim 7.

9. The organic light emitting device of claim 8, wherein the organic film is an emitting layer.

10. A phosphor host compound, represented by the following Chemical Formula 8:

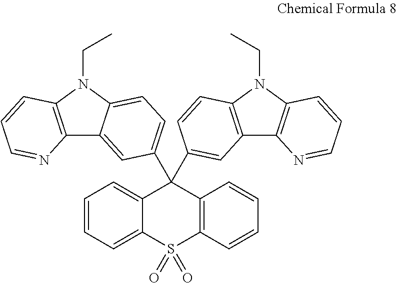 [SMILES: CCn1c2ccc(C3(c4ccc5c(c4)c4ncccc4n5CC)c4ccccc4S(=O)(=O)c4ccccc43)cc2c2ncccc21]

11. An organic light emitting device, comprising an organic film between a pair of electrodes, wherein the organic film comprises the phosphor host compound of claim 10.

12. The organic light emitting device of claim 11, wherein the organic film is an emitting layer.

13. A phosphor host compound, represented by the following Chemical Formula 9:

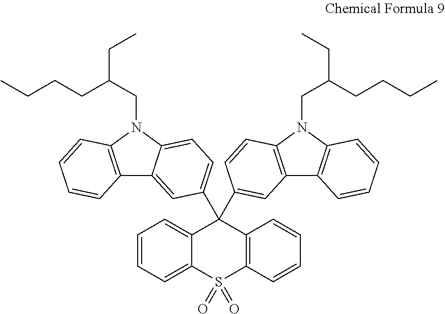 [SMILES: CCCCC(CC)Cn1c2ccccc2c2cc(C3(c4ccc5c(c4)c4ccccc4n5CC(CC)CCCC)c4ccccc4S(=O)(=O)c4ccccc43)ccc21]

14. An organic light emitting device, comprising an organic film between a pair of electrodes, wherein the organic film comprises the phosphor host compound of claim 13.

15. The organic light emitting device of claim 14, wherein the organic film is an emitting layer.

16. A phosphor host compound, represented by the following Chemical Formula 10:

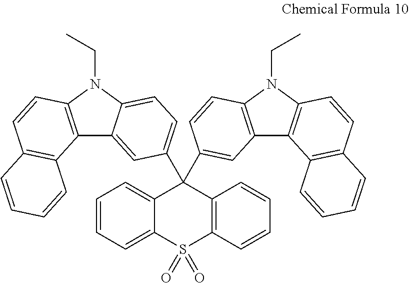 [SMILES: CCn1c2ccc(C3(c4ccc5c(c4)c4c6ccccc6ccc4n5CC)c4ccccc4S(=O)(=O)c4ccccc43)cc2c2c3ccccc3ccc21]

17. An organic light emitting device, comprising an organic film between a pair of electrodes, wherein the organic film comprises the phosphor host compound of claim 16.

18. The organic light emitting device of claim 17, wherein the organic film is an emitting layer.

19. A phosphor host compound, represented by the following Chemical Formula 11:

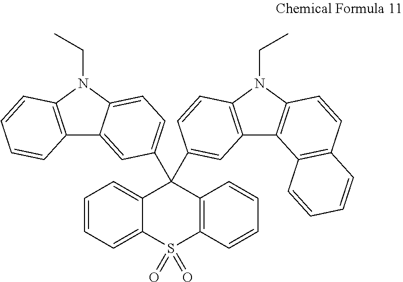 [SMILES: CCn1c2ccccc2c2cc(C3(c4ccc5c(c4)c4c6ccccc6ccc4n5CC)c4ccccc4S(=O)(=O)c4ccccc43)ccc21]

20. An organic light emitting device, comprising an organic film between a pair of electrodes, wherein the organic film comprises the phosphor host compound of claim 19.

8. (canceled)

9. (canceled)
